(19) United States
(12) Patent Application Publication
(10) Pub. No.: US 2025/0163354 A1
(43) Pub. Date: May 22, 2025

(54) CELL CULTURE DEVICE AND CELL CULTURE SYSTEM COMPRISING SAME

(71) Applicant: POSTECH RESEARCH AND BUSINESS DEVELOPMENT FOUNDATION, Pohang-si (KR)

(72) Inventors: Dong Sung KIM, Pohang-si (KR); Hyeon Beom CHONG, Pohang-si (KR); Jun Yeol RHYOU, Pohang-si (KR); Jae Seung YOUN, Pohang-si (KR)

(21) Appl. No.: 18/837,988

(22) PCT Filed: Jul. 21, 2022
(86) PCT No.: PCT/KR2022/010720

§ 371 Date: Aug. 13, 2024

(30) Foreign Application Priority Data
Jul. 21, 2022 (KR) 10-2022-0090337

(51) Int. Cl. C12M 1/32; C12M 1/12; C12M 1/42; C12M 3/00

(52) U.S. Cl. CPC C12M 23/12; C12M 23/48; C12M 25/04; C12M 35/04

(57) ABSTRACT

The present invention may provide a cell culture device and a cell culture system comprising same, wherein the cell culture device comprises: a plurality of upper chambers provided with a porous membrane on the lower side thereof and first accommodation spaces thereinside; a lower chamber configured to provide a second accommodation spaces therein; and a support configured to prevent the plurality of upper chambers from coming into contact with the lower chamber and configured such that the plurality of upper chambers are spaced apart from each other in a horizontal direction by a predetermined distance, wherein the first accommodation spaces and the second accommodation spaces are configured to exchange materials with each other through the porous membrane, and the second accommodation spaces have a material exchange area common to each of the first accommodation spaces provided in the plurality of upper chambers.

### TECHNICAL FIELD

The present disclosure relates to a cell culture device and a cell culture system including the same, and more specifically, to a cell culture device which enables easy exchange of substances secreted or produced during the cultivation of cells or microorganisms cultured in separate spaces, and a cell culture system including the same,.

### BACKGROUND ART

Lab-on-a-chip technology based on microfluidics is expanding into the concept of biomimetic organ-on-a-chip, and is showing potential for application as a new platform for drug response testing. Accordingly, many researchers have recently been actively conducting design optimization research for specific purposes using microfluidic technology. This technology can provide quantitative information on drug screening and drug delivery systems by implementing the properties and functions of different organs in an in vitro environment and administering influencing factors (such as other cells, cell secretions, culture media, and other biochemical substances) that can affect cultured cells.

Meanwhile, in relation to this, Korean Patent Application Publication No. 10-2017-0142729 has been disclosed. This conventional technology is configured to allow cells and culture media to be cultured in independent spaces, and adopts a structure that arranges multiple units under the same culture conditions.

However, this conventional technology generally have only two independent spaces, allowing observation of cell responses to only one influencing factor (such as other cells, cell secretions, chemicals, etc.), and it is difficult to identify the combined effects of multiple factors on cells. In addition, since it relies on simple diffusion phenomena, it is difficult or time-consuming to achieve a uniform concentration distribution of the influencing factors, and it is not easy to control the distribution.

### DISCLOSURE

### Technical Problem

In order to solve the problems of the prior art, the present disclosure provides a cell culture device and a cell culture system including the same, which allows interchange of some substances in independent culture spaces and can control the concentration of the substances at different locations through diffusion and advection.

### Technical Solution

In accordance with the present disclosure, there may be provided a cell culture device comprising: a plurality of upper chambers each of which is provided with a porous membrane on a lower side and has a first accommodation space therein; a lower chamber configured to provide a second accommodation space therein; and a support part configured to support the plurality of upper chambers horizontally spaced apart from each other by a predetermined distance while preventing contact with the lower chamber, wherein the first accommodation space and the second accommodation space are configured to allow exchange of substances through the porous membrane, and the second accommodation space forms a common substance exchange region with each of the first accommodation spaces provided in the plurality of upper chambers.

Further, there may be provided a cell culture system comprising an orbital shaker configured to generate movement in the cell culture device.

Further, there may be provided a cell culture system comprising a rocking shaker configured to generate movement in the cell culture device.

### Advantageous Effects

The cell culture device and the cell culture system including the same according to the present disclosure can culture a culture subject or an influential factor affecting the culture subject in a plurality of independent environments and form a common substance exchange region, thereby enabling the identification of interactions between various combinations of culture subject-culture subject, culture subject-influencing factor, and influential factor-influencing factor.

In addition, the size and concentration distribution of the substance exchange region through diffusion and advection can be controlled, allowing for rapid and systematic analysis of interactions between multiple culture subjects and influencing factors.

### DESCRIPTION OF DRAWINGS

FIG. 1 is a perspective view of a cell culture apparatus according to a first embodiment of the present disclosure.

FIG. 2 is an exploded perspective view of the first embodiment.

FIG. 3 is a cross-sectional view taken along line I-I′ of the first embodiment.

FIGS. 4, 5A and 5B are diagrams illustrating the state of use of the first embodiment.

FIG. 6A and 6B are diagrams illustrating the state of use of a substance exchange region by comparing the first embodiment and the prior art.

FIG. 7A and 7B are diagrams illustrating the gap between an upper chamber and a lower chamber, and the resulting substance exchange region.

FIG. 8A, 8B and 8C are conceptual diagrams illustrating the expansion of the substance exchange region in a cell culture chamber when movement caused by a shaker occurs.

FIGS. 9 and 10 show diagrams illustrating a comparison of substance exchange regions depending on the shape of the side walls of the lower chamber.

FIG. 11 shows diagrams illustrating the substance exchange region according to the size of the lower chamber.

FIG. 12 is a diagram illustrating a modified example of the first embodiment.

FIG. 13 is a perspective view of a cell culture system according to a second embodiment of the present disclosure.

FIG. 14 is a perspective view of the cell culture system according to the second embodiment of the present disclosure.

### MODE FOR DISCLOSURE

Hereinafter, a cell culture device according to embodiments of the present disclosure and a cell culture system including the same will be described in detail with reference to the accompanying drawings. In addition, in the description of the embodiments below, the names of the respective components may be referred to by different names in the art. However, if there is functional similarity and identity between them, even if a modified embodiment is adopted, they can be considered as equivalent configurations. Further, the reference numerals given to the respective components are described for the convenience of explanation. However, the illustrated content in the drawings in which these reference numerals are described does not limit each component to the scope within the drawings. Likewise, even if an embodiment with some modifications to the configuration in the drawings is adopted, if there is functional similarity and identity, it can be considered as an equivalent configuration. In addition, if a component is recognized as obvious to be included in light of the level of ordinary skill in the art, a description thereof will be omitted.

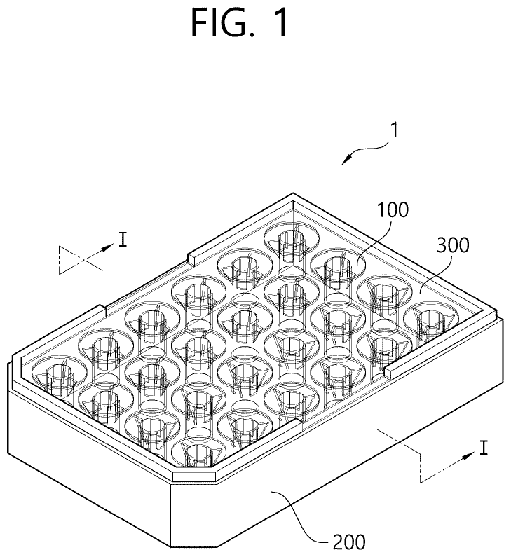

Hereinafter, a cell culture device according to a first embodiment of the present disclosure will be described with reference to FIGS. 1 to 12.

As used herein, the term “culture subject” may be a cell derived from a living organism, or may also be a microorganism. Specifically, it may be a cell derived from tissues such as the heart, lungs, liver, small intestine, large intestine, kidney, nerves, and skin. In addition, the term “microorganism” may be a microorganism that interacts with and can coexist with the human body, such as intestinal microorganisms or microorganisms that live on the skin. Further, the term “substance” may be a substance produced by the culture subject during cultivation in the cell culture device, or may be a substance itself introduced by a user.

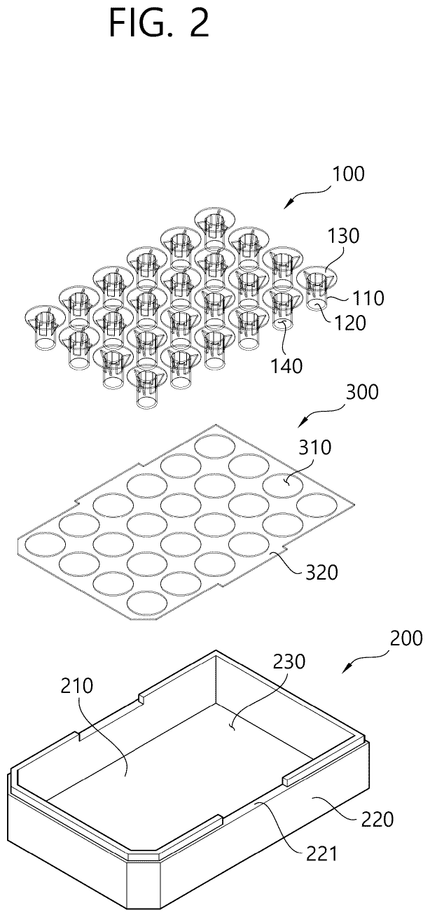

FIG. 1 is a perspective view of the cell culture apparatus according to the first embodiment of the present disclosure, and FIG. 2 is an exploded perspective view of the first embodiment.

Referring to FIGS. 1 and 2, the cell culture device 1 according to the first embodiment of the present disclosure may be configured to create independent culture environments in an upper chamber 100 and a lower chamber 200, while allowing substance exchange between the inside and the outside through one side of the upper chamber 100. In this case, a common substance exchange region between a plurality of upper chambers 100 and the lower chamber 200 may be created.

The cell culture device 1 may be configured to include the upper chamber 100, a support part 300, and the lower chamber 200.

The upper chamber 100 may be formed with an inner wall 110 so that a first accommodation space 140 may be provided on the inner side, and the inner wall 110 may be formed to extend to a predetermined length in a longitudinal direction. As an example, the inner wall 110 may be configured to have a cylindrical shape. A porous membrane 120 may be provided at the bottom of the upper chamber 100 so that substances may be exchanged.

The porous membrane 120 may be configured to block the movement of the culture subject during culture, but allow the movement of substances. The porous membrane 120 may be formed with pores having an inner diameter of several tens of nm to several hundreds of μm. The porous membrane 120 may have a thickness of several μm to several mm.

The upper chamber 100 may be provided with an extension portion 130 extending radially at the upper side. The extension portion 130 may be configured to be stably supported by being fixed on the support part 300 to be described later.

Meanwhile, the configuration of the upper chamber 100 described above is merely an example, and may be implemented in various modified forms with an accommodation space provided on the inside and the porous membrane 120 for substance exchange on one side.

The lower chamber 200 may be provided with a second accommodation space 230 for receiving a culture subject inside, and may be configured to include a side wall 220 and a bottom wall 210.

The bottom wall 210 may be formed to extend from the lower side of the accommodation space to a predetermined area. The side wall 220 may be formed by extending along the perimeter of the bottom wall 210 to a predetermined length in a direction perpendicular to the bottom wall 210, i.e., in a vertical direction.

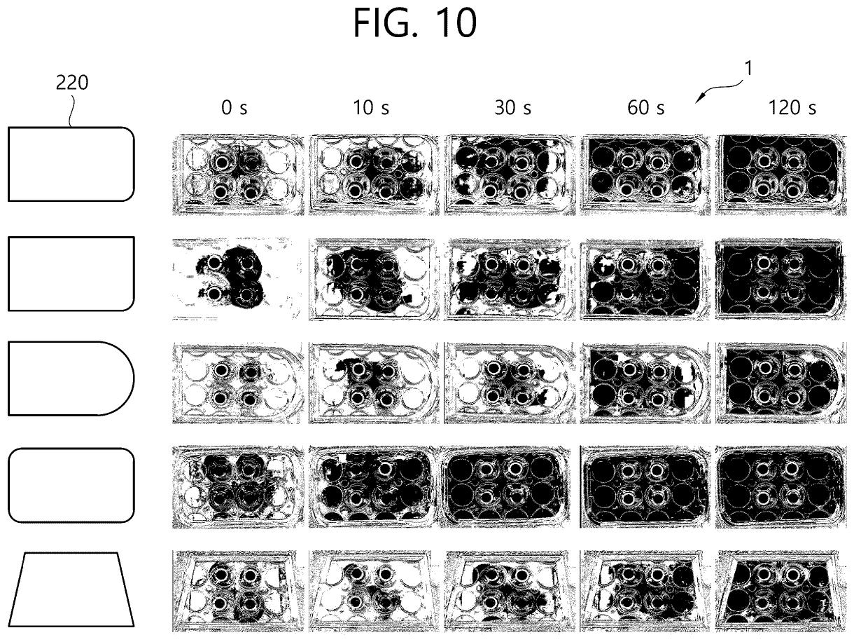

At least a portion of the side wall 220 may be planar, and in FIGS. 1 and 2, the side wall 220 may extend along a generally rectangular path, and may extend along a hexagonal path in which some corners are connected to each other. A recess 221 may be provided on one side of the side wall 220. The recess 221 may be formed from an upper end of the side wall 220 to a predetermined size so that a protrusion 320 of the support part 300 to be described later can be seated therein. However, the configuration of the side wall 220 is merely an example and is not limited thereto, and may be implemented in various modified forms as in the configuration disclosed in FIG. 10 to be described later.

The support part 300 may be configured to support a plurality of upper chambers 100. The support part 300 may be formed with seating holes 310 configured in a predetermined arrangement. The upper chambers 100 described above may be respectively inserted into and supported in the seating holes 310. In this case, the seating holes 310 may be configured to have an inner diameter smaller than the outer diameter of the extension portion 130 of the upper chamber 100 to stably support the upper chamber 100. On both sides of the support part 300, a protrusion 320 extending somewhat horizontally may be provided. The protrusion 320 may be configured to be seated on the upper side of the recess 221 formed in the side wall 220.

Meanwhile, the above-described configuration for the support part 300 to be seated on the lower chamber 200 is merely an example, and may be applied by being modified in various configurations in which the support part 300 can be placed while maintaining the gap between the bottom of the lower chamber 200 and the support part 300.

Although not shown, the cell culture device 1 may further include a cap coupled to the lower chamber 200. The cap helps to isolate the internal and external environments of the cell culture device 1 and minimize the influence of the external environment.

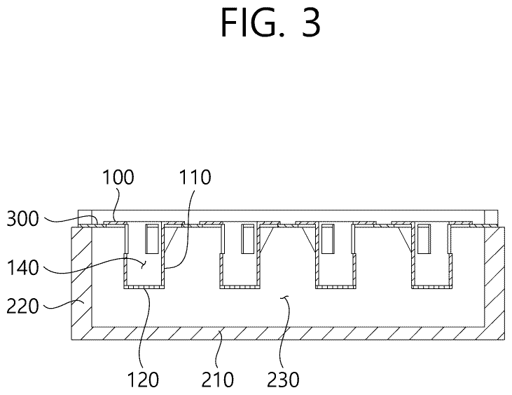

FIG. 3 is a cross-sectional view taken along line I-I′ of the first embodiment.

Referring to FIG. 3, the cell culture device according to the first embodiment of the present disclosure may have a plurality of first accommodation spaces 140 and the second accommodation space 230. A plurality of upper chambers 100 are placed on the support part 300, and each of the first accommodation spaces 140 within the plurality of upper chambers 100 may have a culture environment formed independently from the second accommodation space 230 by the inner wall 110. The second accommodation space 230 may be at least a part of the inner space defined by the bottom wall 210 and the side wall 220 of the lower chamber. In this case, the plurality of first accommodation spaces 140 may be configured to exchange substances with the second accommodation space 230 through the porous membranes, respectively. A culture subject or substance may be accommodated in one side of the first accommodation spaces 140 and/or the second accommodation space 230. When a culture subject is accommodated, an appropriate substance necessary for cultivation of the culture subject, for example, a cell culture medium, may be accommodated together in the first accommodation spaces 140 and/or the second accommodation space 230.

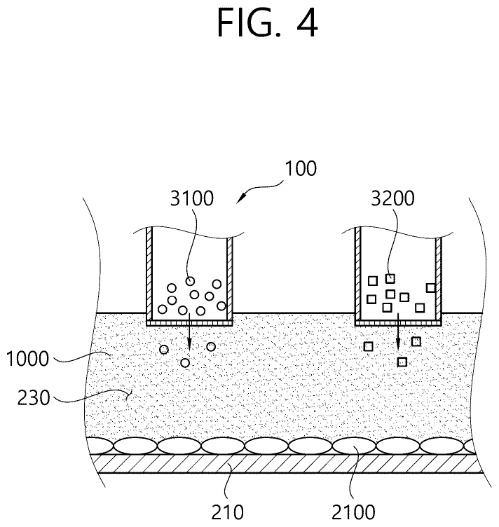

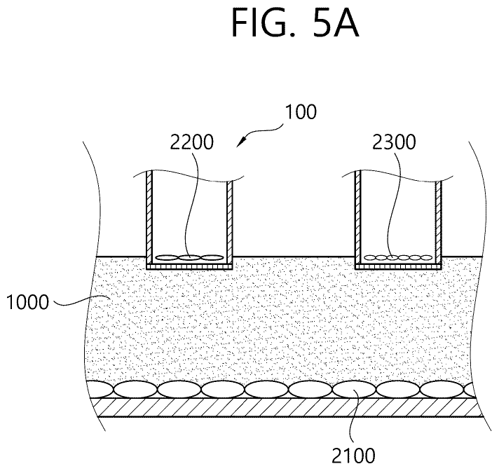

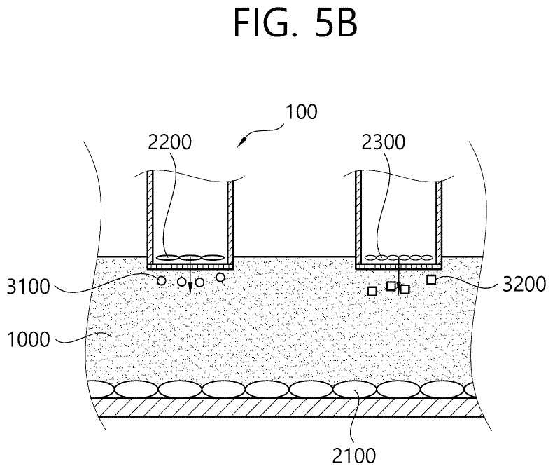

FIGS. 4, 5A and 5B show diagrams illustrating the state of use of the first embodiment. In these drawings, for convenience of explanation, a part of the configuration showing two upper chambers are enlarged and illustrated, and also for convenience of explanation, some elements may be exaggerated or reduced.

Referring to FIG. 4, the two upper chambers 100 accommodate a first substance 3100 and a second substance 3200, respectively. In addition, a cell culture layer on which a first culture subject 2100 is applied may be formed on an upper surface of the bottom wall 210 of the lower chamber. In addition, a cell culture medium 1000 may be accommodated in the second accommodation space 230 to submerge the cell culture layer. In this case, the amount of the cell culture medium 1000 may be determined so that its surface level is higher than the height of the porous membrane. Accordingly, substance exchange may occur between the first accommodation spaces of the upper chambers 100 and the second accommodation space of the lower chamber. At this time, the first substance 3100 and the second substance 3200 may be substances having a size that can pass through the porous membrane and move to the second accommodation space. In this case, the first substance 3100 and the second substance 3200 that have passed through the porous membrane can be diffused again within the cell culture medium 1000 by mixing or dispersion as a result of diffusion and advection.

Referring to FIG. 5A, unlike in FIG. 4, a second culture subject and a third culture subject may be accommodated in each of the upper chambers. Referring to FIG. 5B, the first substance 3100 or the second substance 3200 produced or secreted as the second culture subject 2200 and the third culture subject 2300 are cultured in the respective upper chambers 100, may move into the cell culture medium 1000 in the second accommodation space through the porous membranes, respectively.

In the examples described with reference to FIGS. 4, 5A and 5B, substance exchange centered on the porous membrane in each upper chamber 100 is described. Meanwhile, substances that have moved through the porous membrane can form a common substance exchange region within the cell culture medium 1000. That is, according to the present embodiment, the cell culture medium 1000 in the lower chamber allows for the mixing of various substances moved from the plurality of upper chambers 100.

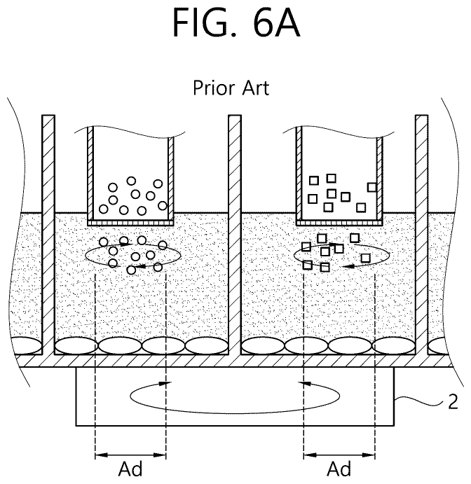

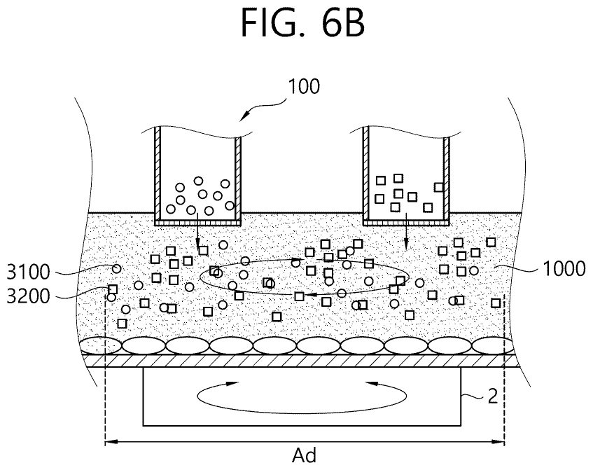

Hereinafter, the substance exchange region formed in the lower chamber will be described in detail with reference to FIGS. 6A to 11.

FIGS. 6A and 6B are diagrams showing the state of use of a substance exchange region Ad by comparing the first embodiment and the prior art.

Referring to FIG. 6A, conventionally, a configuration in which each upper chamber cultures in an independent isolation space has been widely applied. In this case, even when movement occurs due to an orbital shaker 2 widely used in cell culture, the substance exchange region Ad is limited. That is, since the occurrence of advection is minimal, substances that pass through the porous membrane in the upper chamber and move into the cell culture medium are aggregated and do not diffuse widely. In other words, the concentration of the substance has a sharp gradient in the direction away from the porous membrane. That is, the change in concentration according to position in the cell culture medium becomes severe. A large gradient in concentration with position, especially when culturing cells or microorganisms in the upper chamber, may lead to poor diffusion or dilution of the product, resulting in high concentration of the product. This difference in the concentration of the product is not appropriate for simulating the environment in a living body where homeostasis is maintained. Similarly, the actual growth environment of microorganisms is practically like an infinite reservoir from the perspective of microorganisms, so rapid concentration changes due to products are not desirable for cultivation.

Referring to FIG. 6B, in the cell culture device according to the first embodiment of the present disclosure, the substance exchange region Ad of substances moved from one upper chamber can extend beyond the lower side of the adjacent upper chamber. That is, the second accommodation space of the lower chamber can function as a common substance exchange region Ad for substances moved from a plurality of upper chambers. Meanwhile, since the second accommodation space is formed in a relatively very wide area compared to the upper chamber, when movement occurs in the cell culture device by an external orbital shaker 2, the advection (arrow) of the cell culture medium 1000 received in the second accommodation space and the diffusion of the substances occur simultaneously. Therefore, compared to the prior art described in FIG. 6A, the substance exchange region Ad where the first substance 3100 and the second substance 3200 move can be formed over a very wide area. In addition, advection can minimize the aggregation of substances passing through the porous membrane around the membrane. Accordingly, the gradient of concentrations of substances introduced from multiple upper chambers in the substance exchange region Ad can be maintained at a low level.

When the substance exchange region Ad is formed widely as in the present embodiment, the concentration of substances around each porous membrane can be maintained constant, and various substances introduced into the cell culture medium from a plurality of upper chambers can be substantially uniformly distributed within the substance exchange region Ad. This configuration can be utilized for various experiments. For example, when different microorganisms are independently cultured in the upper chambers, the effects of substances produced from the respective microorganisms on cells cultured simultaneously can be determined. In addition, when different cells are simultaneously cultured in the respective upper chambers, it can be utilized in an experiment that analyzes the effects of substances secreted or produced by the cells and passing through the membranes on cells being cultured in the lower chamber.

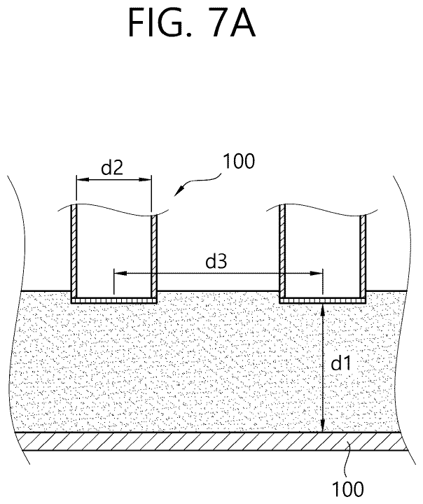

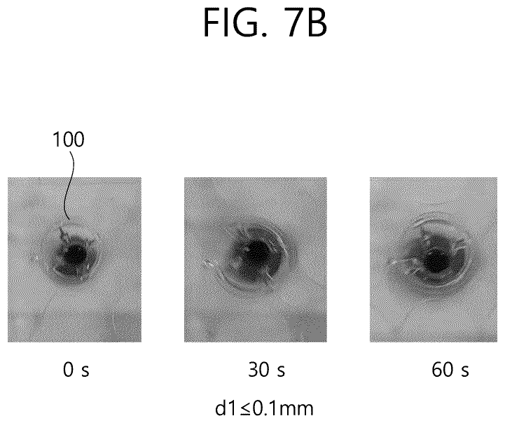

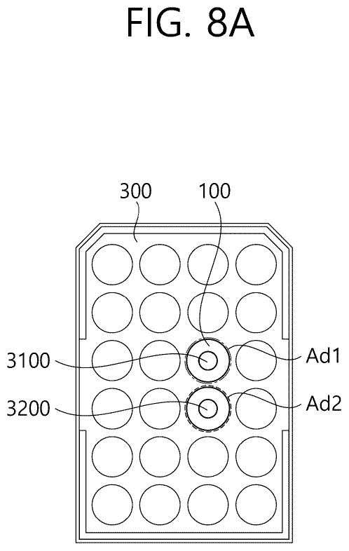

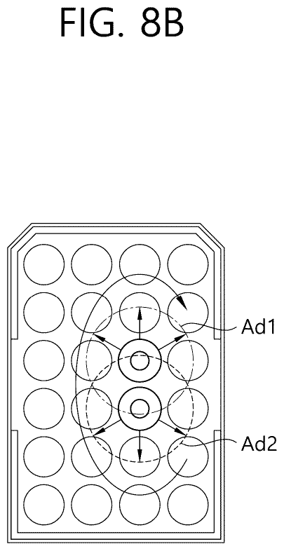

FIGS. 7A and 7B are diagrams showing the gap between the upper chamber and the lower chamber, and the resulting substance exchange region.

Referring to FIG. 7A, the cell culture device according to the first embodiment of the present disclosure is configured to increase the occurrence of advection when the substances accommodated in the second accommodation space are subjected to an external force, for example, an external force caused by an orbital shaker or a rocking shaker. In other words, the cell culture device according to the first embodiment can be configured to minimize factors that suppress the occurrence of advection. As an example, a gap dl between the porous membrane of the upper chamber 100 and the upper surface of the bottom wall 210 of the lower chamber may be 0.1 mm or more. To this end, the vertical length of the upper chamber and the depth of the recess of the side wall of the lower chamber can be determined. In addition, the gap d1 may be determined as a distance at which the bottom surface of the porous membrane and a cell culture layer do not come into contact when the cell culture layer is provided on the bottom surface in the lower chamber. This ensures that the porous membrane does not come into contact with the top of the cell culture layer, which would otherwise prevent the occurrence of advection at the bottom surface of the porous membrane.

Meanwhile, an inner diameter d2 of an inner wall of the upper chamber may be configured to be smaller than a distance d3 between adjacent upper chambers. This is to minimize the resistance to advection caused by the inner wall, as the advection of liquid in the second accommodation space is inhibited when the upper chambers are provided too densely. For example, when the inner diameter d2 of the upper chamber is determined to be 1 cm, the distance d3 between the upper chambers may be 1 cm or more. In this case, the distance between the upper chambers may be determined according to the distance between the seating holes of the support part, and the distance between the seating holes may also be 0.1 cm or more.

Referring to FIG. 7B, when the cell culture device is configured such that the gap d1 between the porous membrane and the lower chamber is 1 mm or less, the substance exchange region formed in the lower chamber can be confirmed. A dye is contained in the upper chamber, and moves into the cell culture medium in the second accommodation space through the porous membrane. However, the dyes that have moved into the cell culture medium mainly move through diffusion even when the cell culture chamber is exposed to the orbital shaker for a certain time (30 seconds, 60 seconds), and the effect of advection is very small. In such cases, it is difficult to expect the effect of uniformly expanding the products or substances that have passed through the porous membrane into the cell culture medium. Therefore, it is preferable that the gap dl between the porous membrane and the lower chamber is 0.1 mm or more.

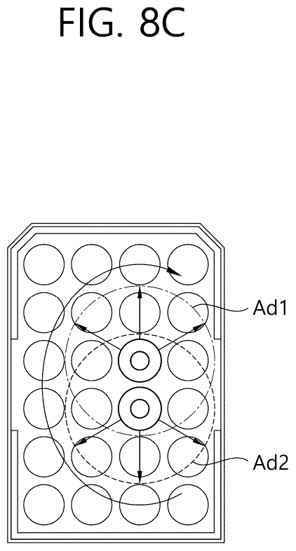

FIGS. 8A, 8B and 8C are conceptual diagrams illustrating the expansion of the substance exchange region in the cell culture chamber when movement caused by the shaker occurs. In these drawings, it is assumed that the first substance 3100 and the second substance 3200 are accommodated in the respective upper chambers, and that the first substance 3100 and the second substance 3200 can move into the lower chamber through the porous membrane. In addition, it is assumed that a culture subject is being cultured on the bottom surface in the lower chamber. Meanwhile, the first substance 3100 and the second substance 3200 may be substances produced by the culture subject being cultured in the upper chambers, respectively.

Referring to FIG. 8A, when no external force is applied, the first substance 3100 and the second substance 3200 that have moved from the upper chambers 100 to the lower chamber form a first substance exchange region Ad1 and a second substance exchange region Ad2 in a somewhat narrow area below the respective upper chambers 100. In this case, the effect on cells being cultured in the lower chamber is determined depending on whether their locations are in the first substance exchange region Ad1 or the second substance exchange region Ad2.

Referring to FIG. 8B, when an external force is applied, advection occurs in the cell culture medium in the second accommodation space. The first substance exchange region Ad1 and the second substance exchange region Ad2 gradually expand due to the advection. In the present embodiment, the substance exchange regions Ad1, Ad2 are described as expanding radially for convenience of explanation, but this is only an example, and flow may occur in various directions due to the advection (curved arrows). In this case, there are a region where the culture subject is affected by either the first substance or the second substance and a region where the culture subject is affected by both the first substance and the second substance simultaneously.

Referring to FIG. 8C, when the exposure time to then external force, e.g., the operating time of the orbital shaker, is increased, the first substance exchange region Ad1 and the second substance exchange region Ad2 are further expanded by the advection, and the region where the culture subject is affected by both the first substance and the second substance can be expanded.

Meanwhile, it is reasonable to consider that the growth of the culture subject and the amount of advection are in a trade-off relationship. That is, if the advection increases, the substance exchange region can be expanded quickly and widely, but on the other hand, the culture subject may move out of position at a certain point, or the growth of the culture subject may be adversely affected by excessive shear force. Therefore, the movement in the cell culture chamber by an external source needs to be limited to a certain level. For example, when the movement in the cell culture chamber is generated by the orbital shaker, it can be operated at conditions of 100 RPM or less to avoid adverse effects on the growth of the culture subject.

Hereinafter, the substance exchange region formed in the second accommodation space according to the shape of the side wall will be described.

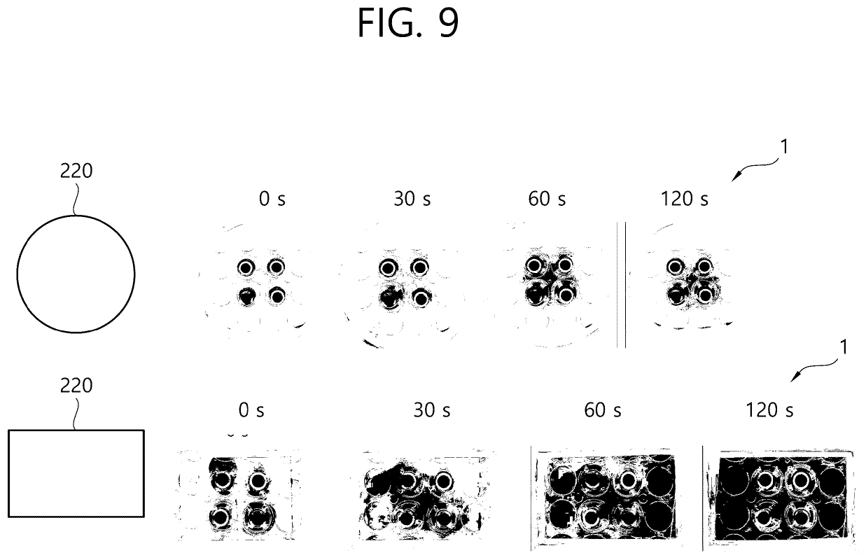

FIGS. 9 and 10 are diagrams illustrating a comparison of substance exchange regions depending on the shape of the side walls of the lower chamber. In these drawings, the cell culture chamber is placed on the orbital shaker, and the expansion of the substance exchange region according to the operating time of the orbital shaker can be confirmed.

Referring to the upper portion of FIG. 9, when the side wall of the lower chamber is formed along a circular path, the expansion of the substance exchange region is not significant even when the orbital shaker is operated for 120 seconds.

On the other hand, referring to the lower portion of FIG. 9, when the side wall is formed along a rectangular path, the substance exchange region is rapidly expanded, and as the orbital shaker is operated for 120 seconds, the substance exchange region is expanded to the entire second accommodation space.

Referring to the leftmost portion of FIG. 10, various shapes of the edge of the side wall are shown. In this case, at least a portion of the side wall may be a flat surface extending in the vertical direction, and in some modified examples, a portion of the side wall is formed as a curved surface.

Referring to the portion of FIG. 10 excluding the leftmost portion, it can be seen that the substance exchange region is expanded when the orbital shaker is operated according to the shapes of the side wall shown in the leftmost portion of FIG. 10. In case that the cell culture chamber moves along a set orbit by the orbital shaker, when at least a portion of the side wall of the cell culture chamber has a flat surface, the advection generated in the cell culture medium in the second accommodation space can be increased.

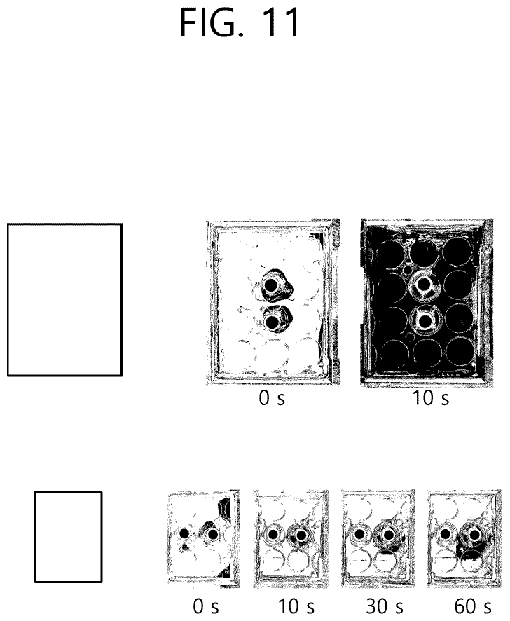

FIGS. 11 shows diagrams illustrating the substance exchange region according to the size of the lower chamber.

Referring to FIG. 11, the change in the substance exchange region according to the horizontal area of the lower chamber can be confirmed. That is, it is preferable that the horizontal area of the second accommodation space is formed somewhat larger than the cross-sectional area of the upper chamber. As an example, the horizontal cross-sectional area of the second accommodation space is preferably 900 mm² or more. However, in order to be stably installed in shakers and cell culture/evaluation devices that are practically widely used, it may be convenient in terms of implementation for the horizontal cross-sectional area of the second accommodation space to be determined as 50,000 mm² or less.

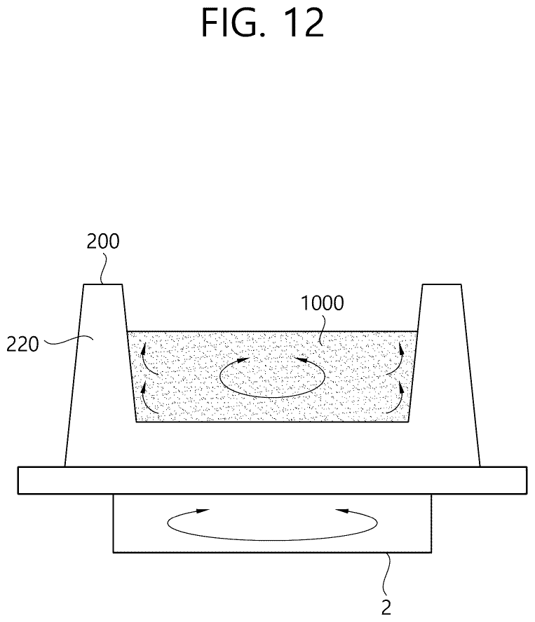

FIG. 12 is a diagram illustrating a modified example of the first embodiment.

Referring to FIG. 12, the side wall 220 of the lower chamber 200 may be configured to have an inner surface inclined along the vertical direction. In particular, the side wall 220 of the lower chamber 200 may be configured such that the normal of the inner surface of the side wall 220 includes an upward vertical component. In this case, when movement is transmitted by the orbital shaker 2, advection may occur not only in the horizontal direction but also in the vertical advection within the cell culture medium 1000. This flow may help the substance exchange region overcome the influence of gravity and expand throughout the entire cell culture medium.

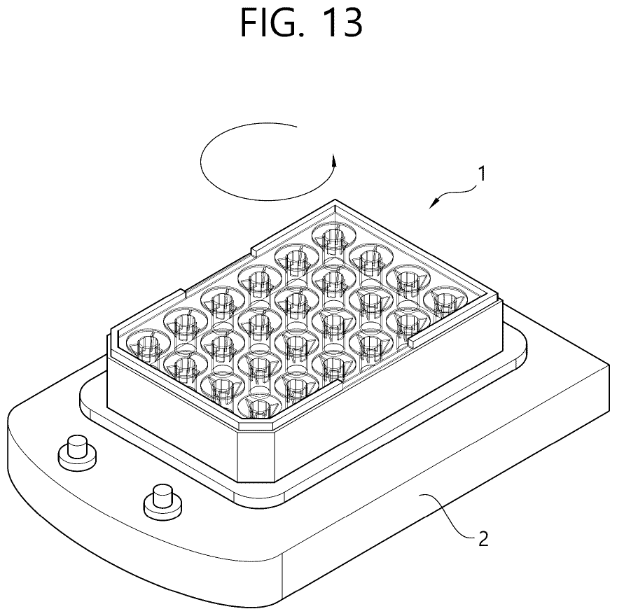

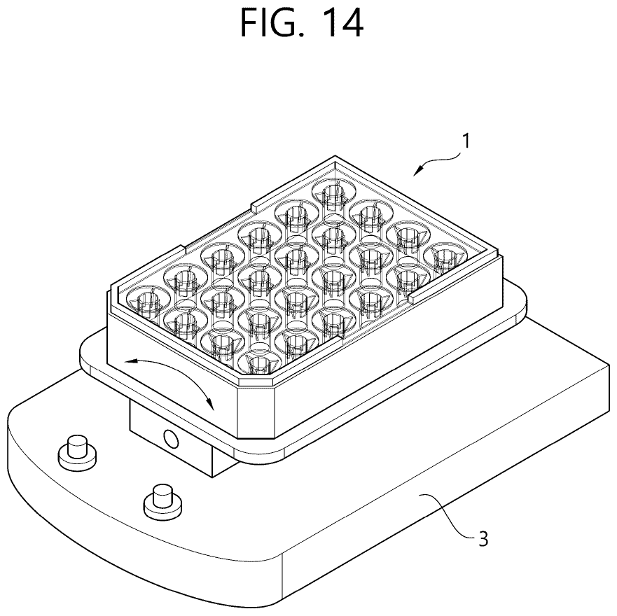

Hereinafter, a cell culture system according to another embodiment of the present disclosure will be described with reference to FIG. 13 and FIG. 14.

FIG. 13 is a perspective view of a cell culture system according to a second embodiment of the present disclosure.

Referring to FIG. 13, the cell culture system according to the second embodiment of the present disclosure may include an orbital shaker 2 and a cell culture device 1. In this case, the cell culture device may be the cell culture device 1 described above with reference to FIGS. 1 to 12.

The orbital shaker 2 is configured to transmit movement to the cell culture device 1 in a state where the cell culture device 1 is seated thereon. The orbital shaker 2 may be operable with parameters of RPM and operating time. In this case, the parameter values may be determined to minimize adverse effects on cells being cultured in the cell culture device 2 and to expand the substance exchange region within the cell culture device 1. As an example, the orbital shaker may be operated at 100 RPM or less. The orbital shaker may then be operated for an appropriate cell culture period to continuously mix or disperse various substances that continuously are introduced from a plurality of upper chambers into a lower chamber as a result of diffusion and advection within the second accommodation space during the cell culture period.

FIG. 14 is a perspective view of the cell culture system according to the second embodiment of the present disclosure.

Referring to FIG. 14, in the cell culture system according to the second embodiment of the present disclosure, a rocking shaker may be used to generate diffusion and advection in the lower chamber of the cell culture device 1. As an example, the rocking shaker may have an upper plate hingedly connected around a front-back axis, and the plate may be inclined to tilt alternately from side to side in a left-right direction. In this case, the rocking shaker may be operated at 100 RPM or less, similar to the operating conditions of the orbital shaker described above, to cause diffusion and flow of substances in the lower chamber of the cell culture device. In the present embodiment, the cell culture chamber of the first embodiment described above may also be used for cell culture.

1. A cell culture device comprising:
a plurality of upper chambers each of which is provided with a porous membrane on a lower side and has a first accommodation space therein;
a lower chamber configured to provide a second accommodation space therein; and
a support part configured to support the plurality of upper chambers horizontally spaced apart from each other by a predetermined distance while preventing contact with the lower chamber,
wherein the first accommodation space and the second accommodation space are configured to allow exchange of substances through the porous membrane, and
the second accommodation space forms a common substance exchange region with each of the first accommodation spaces provided in the plurality of upper chambers.

2. The cell culture device of claim 1, wherein each of the upper chambers is provided such that a bottom surface of the porous membrane is spaced apart from the lower chamber by 0.1 mm or more to allow the substance moved from the first accommodation space to the second accommodation space to travel the predetermined distance or more through diffusion and advection.

3. The cell culture device of claim 2, wherein each of the upper chambers includes:
an extension portion with an expanded outer diameter formed at an upper end; and
an inner wall extending downward from the extension portion to a predetermined length, and
wherein the porous membrane is provided at a bottom end of the inner wall.

4. The cell culture device of claim 3, wherein a separation distance between the inner walls of the adjacent upper chambers is configured to be larger than an inner diameter of the inner wall.

5. The cell culture device of claim 4, wherein the support part includes a plurality of seating holes formed in a predetermined arrangement, and
the seating holes are formed to have an inner diameter smaller than the outer diameter of the extension portion of the upper chamber to allow the upper chamber to be seated.

6. The cell culture device of claim 5, wherein the inner diameter of the inner wall is configured to be 1 cm or less, and a distance between the plurality of seating holes is configured to be 1 cm or more.

7. The cell culture device of claim 5, wherein the second accommodation space is configured to have a horizontal cross-sectional area of 900 mm² to 50,000 mm².

8. The cell culture device of claim 3, wherein a liquid is accommodated in the second accommodation space at a level higher than a height of the porous membrane.

9. The cell culture device of claim 8, wherein cells are cultured in the first accommodation space and/or the second accommodation space.

10. The cell culture device of claim 9, wherein the lower chamber is configured to include a bottom wall and a side wall, and at least a portion of the side wall extends along a plane.

11. The cell culture device of claim 10, wherein a cell culture layer is provided on an upper surface of the bottom wall of the lower chamber, and
the bottom surface of the porous membrane is configured to avoid contact with the cell culture layer.

12. The cell culture device of claim 11, wherein in the lower chamber, the substance exchange region is expanded by the convection to reach at least one of the side wall under operating conditions within 60 seconds by an external shaker.

13. The cell culture device of claim 12, wherein the shaker is an orbital shaker, and
the substance exchange region is expanded within the second accommodation space under operating conditions of the orbital shaker at 100 RPM or less.

14. The cell culture device of claim 12, wherein the shaker is a rocking shaker, and
the substance exchange region is expanded within the second accommodation space under operating conditions of the rocking shaker at 100 RPM or less.

15. The cell culture device of claim 13, wherein when movement occurs in the lower chamber by the external orbital shaker, advection occurs in the liquid accommodated in the second accommodation space by the inner wall of the upper chamber and the side wall of the lower chamber.

16. The cell culture device of claim 14, wherein an inner surface of the side wall is formed at a predetermined angle with respect to a vertical direction to generate fluid flow in an up-down direction within the second accommodation space when movement occurs in the lower chamber by the external orbital shaker.

17. A cell culture system comprising:
a cell culture device; and
a shaker configured to generate movement in the cell culture device,
wherein the cell culture device comprises:
a plurality of upper chambers each of which is provided with a porous membrane on a lower side and has a first accommodation space therein;
a lower chamber configured to provide a second accommodation space therein; and
a support part configured to support the plurality of upper chambers horizontally spaced apart from each other by a predetermined distance while preventing contact with the lower chamber,
wherein the first accommodation space and the second accommodation space are configured to allow exchange of substances through the porous membrane, and
wherein the second accommodation space forms a common substance exchange region with each of the first accommodation spaces provided in the plurality of upper chambers.

18. The cell culture system of claim 17, wherein the lower chamber is configured such that when the cell culture device is moved by the shaker within 60 seconds, the substance exchange region expands to reach at least one of the side walls of the lower chamber.
